(19) United States
(12) Patent Application Publication
(10) Pub. No.: US 2026/0108536 A1
(43) Pub. Date: Apr. 23, 2026

(54) Cannabinoid based nanoplatform composition and methods for treating breast cancer by inhibiting VEGF

(71) Applicant: Cannabis Bioscience International Holdings Inc, Houston, TX (US)

(72) Inventors: Rosangel del Valle Andrades Fuentes, Caracas (VE); Nancy Paola Duarte Delgado, Bogotá (CO); Nancy Rodrigues Das Fontes, Viseu (PT); Stephanie Dona Hartmann, Caracas (VE); Ana Maria Maldonado Vacca, Bogotá (CO)

(73) Assignee: Cannabis Bioscience International Holdings Inc, Houston, TX (US)

(21) Appl. No.: 18/918,386

(22) Filed: Oct. 17, 2024

(51) Int. Cl. A61K 31/00; A61K 9/107; A61K 9/48; A61K 47/10

(52) U.S. Cl. CPC A61K 31/658; A61K 9/1075; A61K 9/4825; A61K 9/4858; A61K 9/4866; A61K 47/10

(57) ABSTRACT

Aspects of the disclosure relate to a composition and methods for treating breast cancer using a cannabinoid-based nanoplatform. The composition comprises phytocannabinoids, including THC, CBD, CBG, and CBC, incorporated into nanoparticles for enhanced bioavailability and controlled release. The method involves administering the composition to patients with breast cancer, particularly stages I and II, to inhibit VEGF pathways and induce apoptosis. The treatment targets estrogen receptor-positive (ER+), progesterone receptor-positive (PR+), HER2-positive, and triple-negative breast cancers. Aspects of the disclosure further provide the production of the composition using nano emulsification techniques to create nanoparticles smaller than 200 nm. This composition offers a novel, targeted approach to reducing tumor growth and metastasis in breast cancer patients.

### FIELD OF THE DISCLOSURE

The technical field of the disclosure relates to medicinal, therapeutic, and healing cannabis and cannabinoids, as well as pharmaceutical cannabis and cannabinoids. The present disclosure pertains to a novel Cannabinoid-Based Nanoplatform Composition as a method for treating breast cancer by selectively inhibiting Vascular Endothelial Growth Factor (VEGF) and tumor angiogenesis through the administration thereof.

The composition leverages advanced nanoplatforms to deliver cannabinoid compounds, enhancing pharmacokinetic properties such as increased bioavailability, which results in greater therapeutic potency and prolonged duration of action.

The combination of cannabinoids described is formulated to serve as an antitumoral and pro-apoptotic therapy, particularly effective in the early stages of breast cancer, including stage I and stage II.

### BACKGROUND INFORMATION

Breast Cancer (BC) is the most common malignancy and leading cause of cancer-related deaths in women worldwide. In 2024, it is estimated that 310,720 new cases of BC will be diagnosed, and 42,250 people will die from the disease. Breast cancer can be classified into four molecular subtypes: luminal A, luminal B, HER2, and triple-negative breast cancer (TNBC), each influencing the treatment strategy. However, current treatments, including endocrine and anti-HER2 therapies, chemotherapy, and radiotherapy, often result in severe side effects and resistance. Metastatic BC has a 5-year survival rate of 29%, dropping to 12% for TNBC (Burguin A 2021).

Angiogenesis, the formation of new blood vessels, is essential for tumor progression. Vascular endothelial growth factor (VEGF) plays a critical role in promoting angiogenesis by stimulating endothelial cell proliferation and survival. VEGF is overexpressed in most human tumors, including breast cancer, and is correlated with tumor size, metastasis, and poor prognosis (Kerbel, 2008; Ferrara and Adamis, 2016). Targeting VEGF can reduce tumor vascularization and inhibit metastasis, making VEGF inhibition a therapeutic strategy.

Cannabinoids, natural compounds from Cannabis sativa, have demonstrated anti-angiogenic properties by impairing the VEGF pathway (Blázquez C 2004). Cannabinoids reduce VEGF levels and suppress tumor vascularization in various cancer models, including gliomas and lung cancer (Blázquez C 2004; Portella G 2007). Additionally, cannabinoids exhibit anti-tumoral and pro-apoptotic effects, inhibiting cancer cell proliferation through pathways involving CB1, CB2, and TRPV1 receptors (Shrivastava A 2011).

Despite substantial advancements in breast cancer therapies in recent years, significant challenges persist, particularly concerning treatment resistance, disease recurrence, and metastasis. Current treatment modalities, while effective for many, often have severe side effects and fail to prevent recurrence in a considerable percentage of early-stage breast cancer patients. Approximately 30% of patients diagnosed with early-stage breast cancer (stages I and II) experience disease recurrence, most commonly in the form of distant metastases, which significantly impacts survival rates and quality of life.

Given these challenges, there is an urgent need for innovative treatment approaches that target novel molecular pathways. The development of such treatments could provide enhanced outcomes, reduce recurrence rates and side effects, and offer improved prognosis for patients with stage I and stage II breast cancer. Novel therapies that focus on specific molecular targets such as VEGF may hold the key to overcoming resistance mechanisms and preventing the progression of the disease, ultimately leading to more durable responses and prolonged survival in breast cancer patients.

Cannabinoids represent a therapeutic option in the treatment of breast cancer through the anti-angiogenic properties exerted by these molecules on the VEGF pathway. Cannabinoid receptors, highly expressed in the most common breast cancer types have also become a therapeutic target in the molecular treatment of breast cancer in early stages I and II, through the administration of cannabinoids that can modulate this molecular pathway on the cancer cell. Therefore, targeting VEGF through a cannabinoid-based approach could offer a novel treatment for breast cancer.

### SUMMARY OF THE DISCLOSURE

This disclosure presents a novel approach to the treatment of breast cancer using phytocannabinoids incorporated into nanoplatform technology. The formulation aims to effectively counteract breast cancer cells and inhibit tumor growth by modulating specific molecular pathways. By combining the therapeutic potential of phytocannabinoids with nanoplatform technology, this disclosure offers a promising strategy to enhance the outcomes of current therapies for breast cancer patients.

The disclosure pertains to a method for producing a formulation for breast cancer treatment by incorporating phytocannabinoids into nanoplatforms. It also refers to a pharmaceutical composition comprising nanoplatforms carrying a combination of phytocannabinoids, including CBD, THC, CBG, and CBC.

The disclosure describes a treatment method using the formulation to inhibit VEGF and induce apoptosis in breast cancer cells. The formulation is administered to patients with HER2+, ER+, PR+, or triple-negative breast cancers (TNBC) in stages I and II. It offers a therapeutic option for aggressive and resistant forms of the disease, with a dosage regimen tailored to effectively target these subtypes and improve patient outcomes.

The disclosure also encompasses a kit containing (a) a composition with nanoparticles carrying phytocannabinoids in a capsule, and (b) instructions for using the composition to treat locally advanced breast cancer. The treatment is for individuals diagnosed with breast cancer at stage I or II, showing HER2, estrogen, or progesterone receptor positivity, or having Triple Negative Breast Cancer (TNBC).

### BRIEF DESCRIPTION OF THE FIGURES

FIG. 1. Mechanisms of Phytocannabinoid Action in Breast Cancer Cells. Top Left: Phytocannabinoids induce cell cycle arrest, preventing cancer cell division and proliferation. Top Center: Tetrahydrocannabinol (THC) decreases vascular endothelial growth factor (VEGF) expression, inhibiting angiogenesis, which is critical for tumor growth. Top Right: Cannabidiol (CBD) induces ER stress, leading to the inhibition of the AKT/mTORC1 pathway, which results in the suppression of cell survival and enhanced apoptosis in breast cancer cells. Bottom Left: Phytocannabinoids promote apoptosis, leading to a reduction in the number of tumor cells, which contributes to a decrease in tumor mass. Bottom Right: Phytocannabinoids inhibit cancer cell migration and reduce the potential for metastasis by affecting the cells' ability to invade surrounding tissues and spread through the vascular system. These combined actions underscore the potential of phytocannabinoids as therapeutic agents in breast cancer treatment.

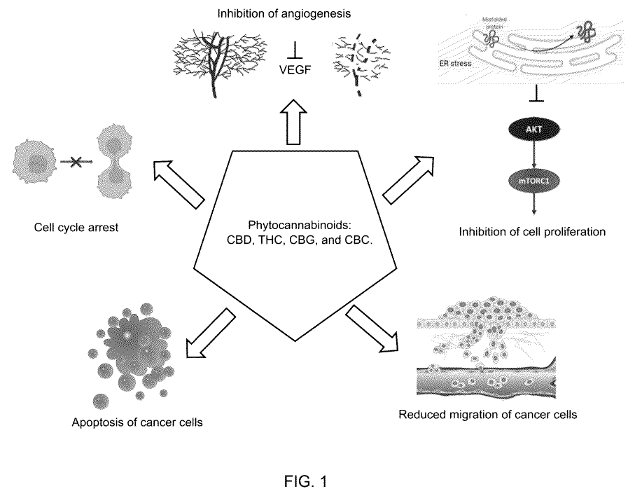

### DETAILED DESCRIPTION

### Definitions

As used herein, the term “individual” refers to a mammal, including humans. An individual includes, but is not limited to, human, bovine, horse, feline, canine, rodent, or primate. In one embodiment, the individual is human. The term “individual’ also includes human patients.

The term “breast” refers to glandular organs located at the top of the chest wall and pectoral muscles, present in both sexes but more developed in females. The breast, particularly the adult female breast, is a complex structure consisting of branching ducts and acini, which group together to form lobules. This ductal-lobular system comprises ducts and acini lined by a dual inner (luminal) epithelial cell layer and an outer (basal) myoepithelial cell layer, resting on a basement membrane and enveloped by stroma.

“Cancer” is an abnormal growth of cells (usually derived from a single abnormal cell). The cells have lost normal control mechanisms and thus can multiply continuously, invade nearby tissues, migrate to distant parts of the body, and promote the growth of new blood vessels from which the cells derive nutrients. Cancerous (malignant) cells can develop from any tissue within the body.

“Breast cancer” is an abnormal growth of breast cells characterized by malignant tumors. Most of the breast malignancies are adenocarcinomas, which constitute more than 95% of breast cancers (Vinay K 2010). Breast cancer can be classified by its histology into in situ carcinoma and invasive (infiltrating) carcinoma, based on their invasion of the basement membrane. Ductal carcinoma in situ (DCIS) is considerably more common than its lobular carcinoma in situ (LCIS) counterpart and encompasses a heterogeneous group of tumors. DCIS has traditionally been further sub-classified based on the architectural features of the tumor which has given rise to five well-recognized subtypes: Comedo, Cribriform, Micropapillary, Papillary, and Solid (Malhotra G 2010).

Breast cancer can also be classified by its molecular markers such as ER, PR, ErbB2 (Her2/neu) and p53. The molecular classification of breast cancer is based on the intrinsic molecular subtypes of breast cancer identified by microarray analysis of patient tumor specimens (Perou C 2000). This classification includes the following subtypes: basal-like, ErbB2+, normal breast-like, luminal subtype A and luminal subtype B.

“Progesterone receptor” or “PR” is a member of the steroid nuclear receptor family of ligand-dependent transcription factors. PR consists of the central portion of the DNA binding domain (DBD), C-terminal ligand-binding domain (LBD) and amino-terminal domain composed of intrinsically disordered protein (Gellersen B 2009). The activated PR recruits diverse functional domains and enzyme activities to the promoter to achieve efficient transcriptional regulation in vivo through interaction with coactivators or corepressors, such as steroid receptor coactivator-1 (SRC-1), SRC2 and SRC-3, cyclic AMP response element-binding protein (CBP/p300) and others.

They will bind the progesterone response element (PRE) of PR and initiate the transcription of target genes. In addition, growth factor-regulated nuclear transcription factors may synergize with agonist-occupied PRs to control the activity of key genes involved in breast cell fate inside the nucleus. PR exists as two predominant isoforms, PR-A and PR-B, which are transcribed from the same gene located on 11q22-q23 by two distinct promoters and exhibit different transcriptional and biological activities as ligand-activated transcription factors.

PR-B is a full-length receptor; PR-A is a truncated form of PR-B lacking 164 amino acids at the N-terminus of the protein. Under progestin-independent conditions, PRA is a more active isoform which is compared to PRB, whereas PR-A is predominantly localized in the nucleus and fails to mediate direct transcriptional events or rapid cytoplasmic changes. Previous research on artificial progesterone-responsive reporter plasmids controlled by the canonical PRE indicated that PR-B is the predominant mediator of progesterone-induced transcriptional activity; however, PR-A has minimal transcriptional activity at the exogenous PRE. PR-A can inhibit the activity of PR-B on ER-chromatin binding activity. The two isoforms are usually co-expressed at similar levels in normal breast, just at 1:1 ratio. In atypical lesions, there was a significant increase in the predominant expression of PRA or PRB, with lesion progression from the normal state to malignancy.

“Estrogen receptor” or “ER” belongs to a family that includes the nuclear ER (nER) and G protein-coupled estrogen receptor 1 (GPER1) (Filardo E 2012). nER is characterized by conserved domain structures, such as the DNA-binding domain (DBD) and the ligand-binding domain (LBD) (Arao Y 2021). Two major nER isoforms, ERα and Erβ, are responsible for regulating the female reproductive system development, preserving bone mass, and protecting the central nervous system, among other physiologically important processes. In humans, the nERs are encoded by two different genes (ESR1 for ERα and ESR2 for ERβ) as a result of gene duplication in the early vertebrate lineage that are located on different chromosomes—ESR1 is located on chromosome 6 and ESR2 on chromosome 14. ERα and ERβ show high homology in the LBD and DBDs, while they differ in the transcription-activating domain (AF-1).

Due to alternative splicing, both receptor subtypes occur in isoforms; five shorter isoforms for ERα, and three shorter isoforms and one longer isoform for ERβ. They are also differentially expressed throughout the body: ERα predominance is shown by the endometrial cells, ovary, and hypothalamus. and outgoing ducts' testicles, while ERβ is expressed mainly in the kidney cells, brain, heart, bones, lungs, intestinal mucosa, prostate, and vascular endothelium. The deregulation of ERα expression and function is closely related to the carcinogenesis process in ovarian, uterine, and breast cancer epithelial cells. On the other hand, ERβ inhibits ERα-mediated transcription and estradiol-induced cell proliferation, which is probably the reason why it is associated with benign forms of breast cancer. However, approximately 75% of breast tumors are ER-positive, and aberrations in the function are associated with ERα. The primary function of both receptors is the downstream regulation of gene transcription upon E2 binding to control the cell proliferation and differentiation activated by the ER-dependent signal transduction (Shanle E 2010).

Progesterone receptor (PR) is an upregulated target gene of estrogen receptor (ER) and its expression is dependent on estrogen levels. There is a mechanistic mutual regulatory interaction between PR and ER expression, where ER regulates PR expression and, in turn, PR modulates ER expression. The presence of PR indicates that the ERα pathway is intact and functionally active. Additionally, PR plays a role in control of several important normal cellular functions, including cell integrity, growth, and proliferation. Although ER and PR are prognostic markers in invasive breast cancer (BC) and key markers for intrinsic subtype classification only ER is used in the clinical setting as a well-established predictive marker of endocrine therapy (ET) and its assessment is mandated in all BC.

The human epidermal growth factor receptor-2 (HER2) receptor (previously called HER2/Neu) is a transmembrane glycoprotein with tyrosine kinase activity that belongs to the epidermal growth factor receptor family. These receptors are essential in controlling epithelial cell growth and differentiation (Klapper L 1999).

HER2 is overexpressed in 15-30% of invasive breast cancers, which has both prognostic and predictive implications. Breast cancers can have up to 25-50 copies of the HER2 gene, and up to 40-100-fold increase in HER2 protein resulting in 2 million receptors expressed at the tumor cell surface. HER2 gene amplification is associated with shorter disease-free and overall survival in breast cancer.

Stage I breast cancer, which is subdivided into “IA” and “IB” depending on the cancer's hormone receptors and HER2-status. In stage IA, the tumor is up to 2 cm in size and the cancer has not spread beyond the breast; there are no lymph nodes involved. In stage IB, there is no tumor in the breast, but small clusters of cancer cells between 0.2 and 2 mm in size are seen in the lymph nodes. A tumor in the breast that is less than 2 cm in size may occur, and small clusters of cancer cells between 0.2 and 2 mm in size are seen in the lymph nodes. If breast cancer is initially classified as stage IB, but is also estrogen receptor- or progesterone receptor-positive, it is classified as stage IA because those two features make it less aggressive. In stage I breast cancer, microscopic invasion (invasive cancer cells no larger than 1 mm) is possible.

Stage II is divided into subcategories IIA and IIB. Stage IIA: There is no tumor in the breast, but tumors (larger than 2 mm) are found in 1 to 3 axillary (underarm) lymph nodes or in lymph nodes near the breastbone (found on sentinel node biopsy). The tumor is 2 cm or smaller and has spread to the axillary lymph nodes. The tumor is 2 to 5 cm in size and has not spread to the axillary lymph nodes.

Staging of breast cancer may be based on a method known to one skilled in the art. Staging of breast cancer may be according to the criteria set forth in American Joint Committee on Cancer (AJCC) Breast Cancer Staging, Eight Edition (2017). For example, the staging of breast cancer may be according to the criteria set forth in Tables 1 and 2.

TABLE 1
TNM Staging for Breast Cancer.
Primary Tumor (T) - Clinical and Pathological

| T Category | T Criteria |
|---|---|
| TX | Primary tumor cannot be assessed |
| T0 | No evidence of primary tumor |
| Tis (DCIS)* | Ductal carcinoma in situ |
| T1 | Tumor ≤20 mm in greatest dimension |
| T2 | Tumor >20 mm but ≤50 mm in greatest dimension |
| T3 | Tumor >50 mm in greatest dimension |
| T4 | Tumor of any size with direct extension to the chest wall |
| | and/or to the skin (ulceration or macroscopic nodules); |
| | invasion of the dermis alone does not qualify as T4 |

Regional Lymph Nodes - Pathological (pN)

| pN Category | pN Criteria |
|---|---|
| pNX | Regional lymph nodes cannot be assessed (e.g., not |
| | removed for pathological study or previously removed) |
| pN0 | No regional lymph node metastasis identified or ITCs only |
| pN1 | Micrometastases; or metastases in 1-3 axillary lymph |
| | nodes; and/or clinically negative internal mammary nodes |
| | with micrometastases or macrometastases by sentinel |
| | lymph node biopsy |
| pN2 | Metastases in 4-9 axillary lymph nodes; or positive |
| | ipsilateral internal mammary lymph nodes by imaging |
| | in the absence of axillary lymph node metastases |
| pN3 | Metastases in 10 or more axillary lymph nodes; or in |
| | infraclavicular (Level III axillary) lymph nodes; |
| | or positive ipsilateral internal mammary lymph nodes |
| | by imaging in the presence of one |
| | or more positive Level I, Il axillary lymph nodes; |
| | or in more than three axillary lymph nodes and |
| | micrometastases |
| | or macrometastases by sentinel lymph node biopsy in |
| | clinically negative ipsilateral internal mammary lymph |
| | nodes; |
| | or in ipsilateral supraclavicular lymph nodes |

Distant Metastasis (M)

| M Category | M Criteria |
|---|---|
| M0 | No clinical or radiographic evidence of distant metastases |
| M1 | Distant metastases detected by clinical and radiographic |
| | means |

TABLE 2
AJCC Cancer Staging Manual, Eighth Edition.

| | | | Then the stage |
|---|---|---|---|
| When Tis . . . | And N is . . . | And M is . . . | group is . . . |
| Tis | N0 | M0 | 0 |
| T1 | N0 | M0 | IA |
| T0 | N1mi | M0 | IB |
| T1 | N1mi | M0 | IB |
| T0 | N1 | M0 | IIA |
| T1 | N1 | M0 | IIA |
| T2 | N0 | M0 | IIA |
| T2 | N1 | M0 | IIB |
| T3 | N0 | M0 | IIB |
| T0 | N2 | M0 | IIIA |
| T1 | N2 | M0 | IIIA |
| T2 | N2 | M0 | IIIA |
| T3 | N1 | M0 | IIIA |
| T3 | N2 | M0 | IIIA |
| T4 | N0 | M0 | IIIB |
| T4 | N1 | M0 | IIIB |
| T4 | N2 | M0 | IIIB |
| Any T | N3 | M0 | IIIC |
| Any T | Any N | M1 | IV |

Suitable methods known in art can be used for assessing a subject to determine whether the cancer is in stage I or II. For example, the size of the tumor and/or tumor markers, usually proteins associated with tumors, can be monitored to determine the state of the cancer. The size of the tumor can be monitored with imaging devices, such as mammography, ultrasound, X-ray, MRI, CAT scans, mammography, PET or via biopsy.

“Tumor markers” are biomolecules, typically proteins, present in or produced by cancer cells or other cells of the body in response to cancer or certain benign (noncancerous) conditions that provide a wide variety of information essential for diagnosing cancer, estimating prognosis, determining effective treatments, assessing how well treatment is working, and checking for recurrence. The most specific tumor markers for breast cancer include CA153, CA2729, CA125, Carcinoembryonic Antigen (CEA), and Circulating Tumor Cells (CTCs).

“Circulating Tumor Cells” or “CTCs” are cancer cells that have detached from a primary tumor, entered the bloodstream, and can potentially travel to other parts of the body to form secondary tumors. CTCs offer a unique opportunity for early detection, monitoring disease progression, and predicting patient outcomes.

The term “tumor-initiating cells” or “TICs” refers to cells present within some tumors that possess the ability to self-renew and proliferate. These cells are sometimes called cancer stem cells (CSCs) and may be observed to share certain characteristics with normal stem cells, including a stem cell-like phenotype and function.

The “sentinel lymph node” is the first lymph node to which cancer cells are most likely to spread from a primary tumor. A sentinel lymph node biopsy (SLNB) involves identifying, removing, and examining the sentinel lymph node(s) to determine whether cancer cells are present. A negative SLNB result suggests that cancer has not yet spread to nearby lymph nodes or other organs, while a positive result indicates that cancer is present in the sentinel lymph node and may have spread to other nearby lymph nodes and possibly other organs.

“Breast Imaging Reporting and Data System” or “BI-RADS” is a classification system developed by the American College of Radiology (ACR) to standardize the interpretation and reporting of mammography and other breast imaging examinations. This system is designed to facilitate communication between radiologists and referring physicians regarding the findings from breast imaging studies, ensuring that both parties have a clear understanding of the results and the need for any subsequent actions or interventions. BI-RADS consists of a series of categories that describe the likelihood of disease presence and the recommended course of action based on the imaging findings. These categories are as follows:
Category 0 (Incomplete): The examination is incomplete or cannot be evaluated.
Category 1 (Negative): No abnormalities found.
Category 2 (Benign Finding): A known benign condition is present.
Category 3 (Probably Benign): An unlikely malignant lesion is detected, and short-term follow-up is suggested.
Category 4 (Suspicious Abnormality): A lesion that is highly likely to be benign but requires further evaluation is seen.
Category 5 (Highly Suggestive of Malignancy): Findings highly suggest malignancy, and biopsy should be considered.
Category 6 (Known Biopsy-Proven Malignancy): A previously diagnosed malignancy is confirmed by imaging.

As described herein, the “active principle” in a pharmaceutical formulation is the therapeutic agent responsible for the desired pharmacological effect. It is the component of the medication that treats the underlying medical condition, in this case, breast cancer.

Cannabinoids are members of a class of terpenophenolic secondary metabolites isolated from Cannabis sativa L. Cannabinoids can exert endogenous functions mediated largely by cannabinoid receptor (CB1 or CB2) signaling, which are G protein-coupled receptors vastly expressed in the membrane of neural, immune, hematopoietic, and glial cells. Over 100 phytocannabinoids have been identified and isolated from the Cannabis sativa L. plant. The most abundant cannabinoids are THC, CBD, CBG, CBN, CBC, THCV, and CBL.

“Cannabinoid receptors” are a class of receptors under the G-protein-coupled receptor superfamily. Their ligands are cannabinoids or endocannabinoids depending on whether they come from external or internal (endogenous) sources. Cannabinoid receptors have a protein structure defined by an array of seven transmembrane-spanning helices with intervening intracellular loops and a C-terminal domain that can associate with G proteins of the Gi/o family (Howlett A 2005).

Evidence obtained thus far has revealed that, as in other cancer types, the endocannabinoid system (ECS) is altered in breast cancer cases, being intimately associated with tumor aggressiveness. Endocannabinoid concentrations and expression levels of cannabinoid receptors (CBs), and of the enzymes responsible for endocannabinoid metabolism, are typically associated with cancer aggressiveness, reinforcing their involvement in cancer development (Śledziński P 2018).

It is known that CB2 is overexpressed in breast cancer and that CB1 is present in significantly lower quantities. CB2 expression is mainly observed in HER2+ tumors and detected in 90% of all HER2+ tumors (Fraguas-Sánchez A 2018). In this case, overexpression of CB2 is linked to a poor prognosis. A correlation has been established between CB2 expression and tumor aggressiveness, as mRNA CB2 levels were higher in ER−/PR− tumors than in ER+/PR+ tumors, as well as in HER2+ tumors than in HER2− tumors, and in high-grade histological tumors than in low-grade histological tumors. On the other hand, CB2 expression on ER+ and ER− tumors is associated with a better prognosis.

“Delta-9-Tetrahydrocannabinol” or “THC” is a phytocannabinoid found in the plant's flowers, leaves, and resin that consists of a tricyclic 21-carbon structure, and has a high affinity to the CB1 receptor, therefore, presents neuroprotective, anti-inflammatory, antiemetics and antiepileptic effects. THC is also responsible for the psychoactive properties of cannabis, mainly through inhalation of cannabis derivatives. The best-established palliative effect of THC is the inhibition of chemotherapy-induced nausea and vomiting, mainly in cancer patients. Oral capsules containing dronabinol (Marinol) or its synthetic analog nabilone (Cesamet) are approved for this purpose. Also, herbal Cannabis has been shown to reduce nausea in most users, when ingested or inhaled. The effect of THC on nausea and vomiting has been confirmed in clinical trials (Meiri E 2007; Jatoi A 2002). Other potential palliative effects of THC in cancer patients include appetite stimulation and pain inhibition. Currently, there is convincing evidence that THC may play a role in the treatment of several types of cancer. In addition to apoptosis and inhibition of proliferation, THC might exert its antitumor effects by inhibiting tumor angiogenesis and metastasis.

“Cannabidiol” or “CBD” is a naturally occurring phytocannabinoid, and a bicyclic compound, being in the split tetrahydropyran ring, virtually has no psychoactive properties. It has powerful antioxidant properties, more potent than ascorbate and -tocopherol. Also, it has notable anti-inflammatory and immunomodulatory effects (Malfait A 2000) Furthermore, sedating, hypnotic, antiepileptic, and anti-dystonic effects have been described. Also, CBD modulates some opioid receptors and can modulate sleep in rats (Murillo-Rodriguez E 2006). Both in vitro and in vivo CBD were able to produce a significant antitumor activity on glioma cells. This antiproliferative effect of CBD was correlated to the induction of apoptosis, which suggests a possible application of CBD as an antineoplastic agent, possibly through a mechanism involving CB2 receptor activation. In another study performed on a panel of tumor cell lines with a variety of plant-derived cannabinoids, CBD was the most potent inhibitor of cancer cell growth, with significantly lower potency in non-cancer cells. It was suggested that the observed effect was due to the capability of CBD to induce apoptosis through cannabinoid receptors, or cannabinoid/vanilloid receptor-independent elevation of intracellular Ca2p and reactive oxygen species.

CBD disrupts cancer cell proliferation by regulating cell cycle progression and interfering with the signaling pathways crucial for cell growth and survival, including the PI3K/AKT/mTOR pathway, the MAPK/ERK pathway, and the NF-κB pathway. Additionally, CBD exhibits anti-angiogenic properties by inhibiting the expression of pro-angiogenic factors and impeding the formation of new blood vessels, thereby hindering tumor progression and metastasis (Martínez Naya, N. 2023).

Go et al. (2020) showed that treatment with CBD demonstrated apoptotic and autophagy events that suppressed the growth and proliferation of head and neck squamous cell carcinomas by promoting the activity of DUSP1 (dual-specificity phosphatase 1), which is capable of inactivating the MAPK isoforms in oncogenic signaling. Zhang et al (2019) observed the anti-proliferative effects of CBD through the upregulation of the ataxia telangiectasia mutated (ATM) gene and the expression of the p21 protein and the downregulation of the p53 protein, leading to a decrease in CDK2 and CCNE, with subsequent G0/G1 phase cell cycle arrest.

“Cannabigerol” or “CBG” is a minor non-psychoactive phytocannabinoid, known a the “stem cell of the cannabis plant” as it precursor cannabigerolic acid (CBGA), is transformed into the main cannabinoid (CBG), δ9-THC, and cannabidiolic acid (CBDA) through oxydocyclase enzymes. CBG was evaluated for antitumor efficacy against mouse skin melanoma cells and showed significant in vivo activity using an methylthiazol tetrazolium (MTT)-based cell viability assay.

Older and recent studies support analgesic, antierythemic, antibacterial, antidepressant, and antihypertensive actions for CBG (Russo E. B 2011). CBG promotes apoptosis, stimulates reactive oxygen species production, and reduces cell growth in CRC cells. Moreover, CBG inhibits the progression of chemically induced colon carcinogenesis and xenograft tumors in vivo (Borrelli et al., 2014). CBG has been proven to be cytotoxic in high dosages on human epithelioid carcinoma cells (Baek S. H 1998), and to inhibit keratinocyte proliferation.

Furthermore, CBG reduced experimental intestinal inflammation, which is relevant given the observation that the risk of developing neoplasia leading to colorectal cancer is significantly increased in ulcerative colitis patients. Pharmacodynamic studies have shown that CBG G interacts with receptors/enzymes involved in carcinogenesis. Specifically, CBG is a weak partial agonist of cannabinoid (CB) 1 and CB2 receptors, inhibits the reuptake of endocannabinoids, is a potent 5-HT1A antagonist and may interact with transient receptor potential (TRP) channels. Among the TRP channels, CBG is a TRPA1, TRPV1, and TRPV2 agonist and, importantly, a potent TRPM8 antagonist (21), a TRP channel known to be involved in the growth of tumor cells (Valero M. 2012).

“Cannabichromene” or “CBC” is a minor non-psychoactive phytocannabinoid. structurally similar to THC and CBD, but differs in its chemical composition, which makes it unique in terms of its potential therapeutic benefits. The scope of CBC research includes a wide range of medical applications, including the management of neuroprotection, the inhibition of NO production, and the improvement in refractory epileptic encephalopathy (CARE-E) (Huntsman, R 2019).

CBC has been associated with anticancer activity and has been proven in vivo and in vitro for its pro-apoptotic effects in prostate carcinoma. Moreover, Anis et al. (2021) identified that cannabis compounds, including CBC, presented cytotoxicity against bladder urothelial carcinoma (UC), the most common urinary system cancer. In the study, the treatment with CBC+THC led to S-phase arrest in the T24 (UC cell line) cell cycle and cell apoptosis, as well it inhibited cell migration, and affected F-actin integrity.

As used herein, “treatment’ or “treating” is an approach for obtaining beneficial or desired results including clinical results. Also encompassed by “treatment’ is a reduction of pathological consequences of breast cancer. The methods of the disclosure contemplate any one or more of these aspects of treatment.

An effective dose can be administered in one or more administrations. In the case of breast cancer, the effective dose of the drug or composition may: (i) reduce the number of breast cancer cells; (ii) reduce tumor size; (iii) inhibit, retard, slow to some extent, and preferably stop breast cancer cell infiltration into peripheral organs; (iv) inhibit (i.e., slow to some extent and preferably stop) tumor metastasis; (V) inhibit tumor growth; (vi) prevent or delay occurrence and/or recurrence of tumor; (vii) relieve to some extent one or more of the symptoms associated with breast cancer; and/or (viii) disrupt (such as destroy) breast cancer stroma.

The treatment for breast cancer depends on the subtype of cancer and how much it has spread outside of the breast to lymph nodes (stages II or III) or other parts of the body (stage IV). For early stages I and IIA, the standard treatment encompasses surgery with breast-conserving surgery (lumpectomy or partial mastectomy) or a modified radical mastectomy (Madden Mastectomy), and adjuvant therapy which could be hormonal therapy (hormone receptor-positive breast cancer), radiotherapy, and/or chemotherapy (American Cancer Society, 2019). However, challenges in the treatment of BC include dealing with treatment resistance and recurrence. Indeed, 30% of early-stage BC have recurrent disease, mostly metastases.

Adjuvant therapy for breast cancer is typically administered after surgery to eliminate any residual cancer cells and reduce the risk of recurrence. This therapy includes options like cytotoxic chemotherapy and hormone therapy. For hormone receptor-positive breast cancer, adjuvant treatments have shown significant improvement in disease-free survival and overall survival. Studies have demonstrated that adding adjuvant chemotherapy, particularly using combinations like CMF (cyclophosphamide, methotrexate, and fluorouracil) or anthracycline-based regimens, provides long-term benefits. In hormone receptor-positive breast cancer patients, chemotherapy in combination with endocrine treatments such as tamoxifen has been linked to a 50% reduction in mortality over 15 years.

Neoadjuvant therapy, given before surgery, aims to shrink the tumor to make surgery easier and more effective, sometimes enabling breast-conserving surgery instead of mastectomy. Traditionally, neoadjuvant therapy is recommended for patients with more advanced breast cancer, such as those with larger tumors or positive lymph nodes. It is also increasingly used in stage II cancers to facilitate less invasive surgery. Studies have highlighted that neoadjuvant therapy, especially with chemotherapy, can reduce tumor size significantly and has been linked to improved survival outcomes when pathologic complete response (pCR) is achieved. In HER2-positive and triple-negative breast cancers, neoadjuvant therapy is particularly effective.

The nanoplatforms are formed by Nanoparticles. “Nanoparticle” is a particle of matter with a diameter of one to one hundred nanometers (nm). The “Nanoplatforms” are engineered nanostructures designed to deliver drugs, genes, or other therapeutic agents in a targeted and efficient manner. They can enhance the bioavailability and efficacy of treatments, often by utilizing their unique properties at the nanoscale, such as increased surface area, improved solubility, and the ability to penetrate biological barriers. Nanoplatforms can include various materials, such as liposomes, dendrimers, and nanoparticles, and are often used in biomedical applications, including drug delivery, imaging, and diagnostics.

Due to their tiny size and distinctive physical and chemical characteristics, NPs represent advantages in pharmaceutical formulation and cancer treatment. The advantages include penetration of biological barriers and access to intracellular targets, reduced risk of toxicity or immune reaction, target specificity which can increase efficacy and safety of drug delivery, better stability for long storage and transportation, and increased bioavailability of compounds delivered. In cannabinoid formulations, nanoparticles can overcome physicochemical challenges, including the lipophilic nature of cannabinoids poses challenges for their formulation and delivery. Nanoparticles, especially lipid nanoparticles, can address these challenges by improving the solubility and bioavailability of cannabinoids, making them more suitable for oral administration and other routes.

The nanoemulsions are produced by high-energy, nano-emulsification methods which are driven by mechanical devices that promote high-intensity cavitation in pre-mixed formulations and are generally more appropriate for the commercial production of nanoemulsions. Cavitation provides high-shear forces that break up and disperse oil droplets to form stable nanoemulsions. High-pressure homogenization (HPH), microfluidization, and ultrasonication are umbrella terms encompassing nano-emulsification methods to form a nanoemulsion. An HPH device utilizes high pressures to force pre-emulsified formulations into an interaction chamber consisting of microchannels that impinge onto each other to promote cavitation (Leibtag and Peshkovsky, 2020).

The nanoparticles detailed here can be part of a composition that incorporates other agents, excipients, or stabilizers. To enhance stability by augmenting the negative Zeta potential of nanoparticles, one or more negatively charged components may be introduced. Examples of such negatively charged components encompass bile salts derived from bile acids such as cholic acid, chenodeoxycholic acid, taurocholic acid, glycochenodeoxycholic acid, taurochenodeoxycholic acid, lithocholic acid, ursodeoxycholic acid, dehydrocholic acid, and others; phospholipids including egg yolk-derived lecithins, which include various phosphatidylcholines and other phospholipids like dimyristoylphosphatidylcholine (DMPC), dioleoylphosphatidylcholine (DOPC), distearoylphosphatidylcholine (DSPC), and hydrogenated soy phosphatidylcholine (HSPC), and related compounds. Additionally, negatively charged surfactants or emulsifiers, such as Sodium cholesteryl Sulfate, are also suitable as additives.

“Gelatin” is a protein molecule hydrolyzed from collagen, which is highly biocompatible and biodegradable under physiological conditions. Gelatin is a highly responsive material that can respond to several environmental changes which include pH, temperature, and other biological signals. Gelatin capsules can control drug dose, effectively improve drug utilization, and enhance the convenience of drug taking and storage capacity. With the development of nanomaterials, gelatin-based drug delivery carrier materials have developed from macroscopic capsules to Drug Delivery Nanosystems (DDnS) such as gelatin-based nanospheres, hydrogels, nanogels, and nanofibers. Gelatin-based drug delivery carrier materials have attracted extensive attention in the field of pharmaceutical medicine.

Based on studies in breast cancer, lung cancer, and skin cancer, gelatin is a kind of drug carrier material with great potential and responsiveness to cancer treatment, which can be used for targeted drug delivery to cancer sites.

As used herein, “angiogenesis” consists in forming new blood capillaries from existing vessels and is an important mechanism for supplying nutrients to distant cells from existing blood vessels. It plays a critical role in the development of new blood vessels from pre-existing ones, which is essential during embryonic development, wound healing, and the formation of new tissues. Angiogenic endothelial cells undergo a complex sequence of events including the secretion of metalloproteases and other matrix-degrading enzymes, cell migration into the newly created space, endothelial cell division and proliferation, and vessel formation. These are well-regulated processes involving several stimulators such as vascular endothelial growth factor (VEGF).

VEGF is a member of a family of proteins including VEGF-B, VEGF-C, VEGF-D, VEGF-E (virally encoded), and placental growth factor (PIGF) (reviewed in Ferrara and Adamis, 2016), capable of promoting the growth of vascular endothelial cells (ECs) derived from arteries, veins, and lymphatics and can act as a survival factor for ECs by preventing apoptosis.

Angiogenesis contributes to the pathology of several diseases, including tumor progression (Carmeliet and Jain, 2000). This is because angiogenesis provides nutrients that maintain the viability of diseased tissue. Tumor-associated angiogenesis allows the tumor to maintain its growth advantage and also facilitates metastatic spreading by establishing connections to the existing vasculature.

VEGF is often overexpressed by tumor cells, promoting the growth of new blood vessels that supply nutrients and oxygen to the tumor, facilitating its growth and spread. Thus, VEGF is a significant target for anti-angiogenic therapies aimed at inhibiting tumor growth by blocking the formation of new blood vessels. Experimental and clinical studies revealed that VEGF is the predominant angiogenic factor in breast cancer (Ribatti et al., 2016).

Overexpression of VEGF occurs frequently before the invasion of breast cancer cells (Schneider and Sledge, 2007). advanced stage of breast cancer (Ribatti et al., 2016). Studies have also found an inverse relationship between VEGF expression and overall survival (OS) in both node-positive and node-negative diseases (Ribatti et al., 2016).

Angiogenesis in breast carcinoma is regulated by VEGFR-2, VEGFR-3, VEGF-D, and VEGF-C (Eroglu et al., 2017). Expression of VEGF-D was associated with lymph node metastasis in breast cancer tissues (Eroglu et al., 2017). Besides VEGF, multiple pro-angiogenic factors are expressed by invasive human breast cancer including TGF-β1, pleiotrophin, acidic and basic FGF placental growth factor, and PDGF. High microvessel density was further associated with invasive carcinoma and correlated with a greater likelihood of metastatic disease and shorter OS in breast cancer patients.

VEGF secreted by tumor cells and surrounding stroma stimulates the proliferation and survival of endothelial cells, leading to the formation of new blood vessels, which may be structurally abnormal and leaky. VEGF mRNA is overexpressed in the majority of human tumors and correlates with invasiveness, vascular density, metastasis, recurrence, and prognosis (Kerbel, 2008).

The complex interactions between tumor angiogenesis, VEGF expression, and cancer development represent therapeutic targets to treat cancer and prevent metastasis. VEGF signaling in cancer cells is responsible for their resistance to apoptotic stimuli and their migration and invasion (Mercurio A 2005). VEGF is highly up-regulated in breast cancer. Compared with normal or benign breast tissues, breast cancer showed higher levels of VEGF transcripts (Rice A 2002).

Approximately 72-98% of breast cancer cases are positive for VEGF by immunohistochemistry (IHC). VEGF expression in breast tumors is correlated with large size, high histologic grade, estrogen receptor (ER) negativity, progesterone receptor (PR) negativity, human epidermal growth factor receptor-2 (HER2) over-expression, and lymph node metastasis. In animals, anti-VEGF therapy inhibits the growth of breast tumors, reduces tumor microvessel density, and limits the infiltration of tumor-associated macrophages. VEGF and their receptors have been identified as a strategic target of modern cancer therapies mostly by inhibition of the VEGF/VEGFR signaling pathway.

VEGF is present exclusively in angiogenic endothelial cells, so targeting VEGF will be less likely to affect quiescent endothelial cells and therefore adverse side effects such as bone marrow suppression, gastrointestinal symptoms, or hair loss that are characteristic of standard chemotherapy treatment can be avoided.

In-vivo and in-vitro research on cannabinoids have shown that they exert anti-angiogenic properties. Cannabinoid treatment impairs the VEGF pathway in mouse gliomas by blunting VEGF production and signaling (Blázquez C 2004). Cannabinoid-induced inhibition of VEGF expression and VEGFR-2 activation also occurred in cultured glioma cells, indicating that the changes observed in vivo may reflect the direct impact of cannabinoids on tumor cells. Moreover, a depression of the VEGF pathway was also evident in two patients with glioblastoma multiforme (Blázquez C 2004).

Cannabinoids have significantly affected vascular endothelial growth factor (VEGF), a critical proangiogenic regulator (Blázquez C 2004). The administration of Δ9-THC, 2-methyl-2′-F-anandamide (Met-F-AEA), WIN-55,212-2, and (6aR, 10aR)-3-(1-dimethylbutyl)-6a,7,10,10a-tetrahydro-6,6,9-trimethyl-6H-dibenzo[b,d] pyran (JWH-133) reduced VEGF levels in numerous cancer cell lines (McDougall S 2006; Blázquez C 2004). In particular, evidence shows that Δ9-THC may potentially lead to reduced VEGF production in lung cancer cell lines SW1573 and A549 and decreased vascularization of A549 cell lines in immunodeficient mice with tumor xenotransplantation (Portella G 2007).

“Apoptosis” is defined as the process of programmed cell death, which is crucial for maintaining the balance of cells within an organism. Apoptosis serves as a defense mechanism against cancer by removing cells that might otherwise accumulate genetic defects and transform into malignant tumors. This process is highly regulated and involves biochemical events leading to characteristic changes in cell morphology, including blebbing, cell shrinkage, nuclear fragmentation, chromatin condensation, DNA fragmentation, and mRNA decay. Apoptosis can be triggered through two main pathways: the intrinsic pathway, where the cell initiates its death in response to stress, and the extrinsic pathway, where the cell dies upon receiving signals from other cells. Both pathways ultimately activate caspases, proteases that degrade proteins and initiate cell death.

Cannabinoids exert anti-tumoral and proapoptotic properties. The antiproliferative effects of phytocannabinoids were demonstrated for the first time in 1975, by Munson et al., who showed that delta-9-tetrahidrocannabinol (THC) and cannabinol (CBN) inhibit lung adenocarcinoma cell growth.

The primary antitumor effects of cannabinoids rely on cell cycle arrest by inhibiting the expression of growth factors and induction of apoptosis. Until now, the actions of cannabinoids have been identified in different tumors, including gliomas, melanomas, lymphomas, breast cancer, skin cancer, lung carcinoma, liver cancer, pancreatic cancer, colon cancer and prostate cancer (Grimaldi C 2011).

A study evaluating the effects of anandamide (AEA), an endogenous cannabinoid, on breast cancer, specifically using the MCF-7 cell line, found that anandamide significantly inhibits both the adhesion and migration of these cells, which are critical processes in cancer metastasis. The mechanisms underlying these effects appear to involve cannabinoid receptors (CB1/CB2) and vanilloid receptors (TRPV1), as blocking these receptors reduces the inhibitory impact of AEA. This suggests that anandamide could potentially interfere with the metastatic spread of breast cancer by impairing the cancer cells' ability to adhere to extracellular matrices and migrate. The research highlights the potential of anandamide as a therapeutic agent for targeting breast cancer metastasis, warranting further exploration into its anti-metastatic properties (Grimaldi et al., 2006).

CBD (cannabidiol) has been the most studied phytocannabinoid in triple-negative breast cancer (TNBC). It has been reported that this phytocannabinoid reduces the proliferation of MDA-MB-231 cells through the direct activation of TRPV1 receptors and possibly through other yet uncharacterized CBD targets (Ligresti A 2006). However, it was also proposed that CBD induces apoptosis in this cell model through CB1, CB2 and TRPV1 receptors. This effect is mediated by endoplasmic reticulum stress and inhibition of the AKT/mTOR pathway, which ultimately culminates in autophagy and mitochondria-driven apoptosis (Shrivastava A 2011). The induction of apoptosis by CBD in MDA-MB-231 cells was also recently verified by Sultan et al., where CBD inhibited cell survival and induced apoptosis, favored by an interplay among PPAR, mTOR and cyclin D1 (Sultan A 2018). The minor phytocannabinoids CBN and CBG also showed interesting results in TNBC. In MCD-MB-231 and MDA-MB436 cells, both compounds reduced cell viability and cell migration through a process probably involving a decrease in Id-1 expression (McAllister S 2007). Recently, it was also verified that CBD can induce endoplasmic reticulum stress, which leads to cell death, in MCF-7 cells by activating TRPV1 and the consequent increase in Ca2+ and ROS levels (De la Harpe A 2021). Regarding minor phytocannabinoids, CBG (cannabigerol) decreased MCF-7 cell proliferation (Ligresti A 2006).

Ligresti et al. (2006) investigated the antitumor activities of other plant cannabinoids, including cannabigerol, which followed CBD in antitumoral potency and exhibited inhibition of cancer cell growth. In the study, the cannabinoids inhibited the growth of xenograft tumors obtained by s.c. injection into athymic mice of human MDA-MB-231 breast carcinoma or rat v-K-ras-transformed thyroid epithelial cells and reduced lung metastases deriving from intrapaw injection of MDA-MB-231 cells. Through several experiments on its possible cellular and molecular mechanisms of action, researchers found CBD and CBG to activate TRPV1 receptors and/or inhibit anandamide inactivation to some extent.

### Pharmaceutical Compositions

In this embodiment, the phytocannabinoids THC, CBD, CBG, and CBC are obtained from the Cannabis sativa L. plant. The formulation may be designed for oral, inhalatory and subdermal administration and may be presented as capsules, tablets, oral drops, essential oil, transdermal patches or gummies, depending on the desired method.

In this embodiment, the composition includes cannabinoids at a concentration ranging from 20% to 40%. The formulation also contains stabilizing solubilizing agents, emulsifying agents, and preservants present at a concentration ranging from 0.1% to 5%.

In one embodiment, the emulsifying system utilizes emulsifying agents may comprise but not limited to PEG-400, Sorbitan, Polysorbate, Lecithin, Sodium Lauryl Sulfate and Poloxamers. The lipids agents used may comprise but not limited to Medium Chain Triglycerides (MCT oil), Mineral Oil, Olive oil and combinations thereof. In one embodiment, the preservants may comprise but not limited to Vitamin E, Vitamin C, EDTA and combinations thereof.

In one preferred embodiment, the formulation comprises the following concentrations: THC at 1.00-2.00%, CBD at 7.00-14.00%, CBG at 6.00-12.00%, and CBC at 6.00-12.00%. In one embodiment, the medium-chain triglycerides are present at 24%-35.80%, which is highly beneficial for the dissolution of cannabinoids.

In one embodiment, the emulsifier used is Sorbitan 80 at 1.4%-2.8% and Tween 80 at the same proportion. In this formulation, PEG 400 will function as an auxiliary in the emulsification process of the cannabinoid present at 0.67-1.33%. In another embodiment, during the preparation process, Span 80, Tween 80 and PEG 400 are incorporated as surfactant systems.

In one embodiment, vitamin E is present at a dose of 0.07%-0.10%. Vitamin E is incorporated to protect the cannabinoids from degradation and improve their absorption and bioavailability. In one embodiment, the formulation contains 29.00% to 39.00% of an aqueous phase vehicle, such as saline phosphate buffer or purified water.

In this embodiment, nanoplatform compositions include nanoparticles with a mean diameter of less than 200 nm. These particles may be delivered in a sustained-release capsule for optimized therapeutic effects in breast cancer treatment. In this embodiment, high-energy nano-emulsification methods are used to create stable nanoemulsions for effective delivery.

In this embodiment, the phytocannabinoid-based pharmaceutical composition is designed for multiple routes of administration and therapeutic applications which may include oral, sublingual, intrarectal, and injectable options, offering flexibility in treatment and ensuring precise control over dosage and bioavailability to meet diverse patient needs.

In this embodiment, the phytocannabinoid-based composition is formulated into oral dosage forms, such as tablets, capsules, oral drops or solutions, designed for ingestion and gastrointestinal absorption. These forms may include immediate-release, sustained-release, or controlled-release options to regulate cannabinoid bioavailability over time.

In this embodiment, sublingual presentations such as drops, sprays, tablets or films are developed for rapid absorption through the mucous membranes, bypassing first-pass metabolism and providing quicker therapeutic effects. In one embodiment, the formulation is provided as injectable solutions or suspensions, designed for intravenous, intramuscular, or subcutaneous administration. These forms offer precise and immediate delivery in settings where rapid cannabinoid action is needed.

In this embodiment, the pharmaceutical composition may further include various excipients or carriers to enhance stability, bioavailability, and patient acceptability. These excipients could include solubilizers, stabilizers, preservatives, bioavailability enhancers, and flavoring and sweetening agents.

In various embodiments, the composition may vary in the concentration of cannabinoids. Additionally, the ratio of different cannabinoids may be varied to optimize the therapeutic profile while maintaining efficacy. In one embodiment, the formulation may combine two or more cannabinoids in specific ratios to produce a synergistic effect, where the combined therapeutic action is greater than the effect of individual cannabinoids alone.

In this embodiment, various delivery enhancements are employed to improve the efficacy and targeting of cannabinoid compositions. Methods may include liposomal encapsulation, nanoparticles and targeted delivery systems.

In this embodiment, the cannabinoid pharmaceutical composition may also include other therapeutic agents to enhance the efficacy of the cannabinoids or provide complementary therapeutic benefits. Other therapeutic agents may include non-cannabinoid pharmaceuticals, such as analgesics, or anti-inflammatories. Additionally, the composition may be combined with herbal extracts, incorporating other plant-derived compounds such as terpenes or flavonoids, which may enhance the therapeutic potential of cannabinoids through the entourage effect.

### Method of Production of the Nanoplatforms/Nanoemulsion

The production of nanoplatforms or nanoemulsions involves various methodologies that can be adapted to create the desired size, stability, and functionality of the final product. These methods can include multiple stages, the use of specific excipients, and carefully controlled conditions such as temperature, pressure, and mixing techniques. Below, different variations in the production process are explored.

In this embodiment, various production techniques may be employed to create nanoemulsions, depending on the desired characteristics and application. These methods may include high-pressure homogenization, ultrasonication, microfluidization, or solvent evaporation/precipitation techniques.

In this embodiment, the production of nanoemulsions may involve high-pressure homogenization. This method utilizes high shear forces to break down larger particles into nanoscale sizes. The pressure and number of cycles can be adjusted to achieve the desired particle size and stability.

In this embodiment, ultrasonication may be employed as the production method, where ultrasonic waves create cavitation to disrupt droplets or particles into nanoscale sizes. The sonication time, power, and frequency may be varied to control the properties of the resulting nanoemulsions.

In this embodiment, microfluidization may be used in the production process. The reactants are passed through microchannels at high velocities, creating uniform and stable nanostructures. The flow rate, pressure, and channel design can be modified to control the particle size and distribution of the nanoemulsions.

In this embodiment, nanoemulsions may be produced through solvent evaporation or precipitation techniques. An organic solvent containing the active ingredients is emulsified in an aqueous phase, followed by solvent evaporation, which leads to the formation of nanoparticles. The solvent choice, emulsifying agents, and evaporation conditions (e.g., temperature, pressure) may be adjusted to achieve the desired characteristics.

Excipients, Surfactants, and Stabilizers: The choice of reactants, surfactants, and stabilizers plays a crucial role in the production process, as these components determine the stability, size, and functionality of the nanoplatforms or nanoemulsions:

Surfactant Selection: Various surfactants may be used to stabilize the nanoemulsion, and their concentration and type can vary depending on the formulation. Nonionic, cationic, or anionic surfactants can be employed, and the hydrophilic-lipophilic balance (HLB) of the surfactant will influence the emulsification process and the size of the nanoparticles.

Excipients for Functionalization: The production method may involve incorporating excipients that modify the surface of the nanoplatforms, allowing for targeted delivery or enhanced interaction with biological systems. These excipients could include stabilizers, antioxidants, preservatives, oil phase vehicles and aqueous vehicles.

Use of Polymers or Lipids: In one embodiment, polymers of lipids are used as the primary building blocks for the nanoplatforms. The production method may involve varying the type and molecular weight of the polymers, or the chain length and saturation of the lipids, to influence the mechanical stability and release properties of the nanoparticles.

Process Conditions (Temperature, Pressure, Mixing): The conditions under which the nanoplatforms or nanoemulsions are produced can greatly affect the final properties, and these conditions can be adjusted for different formulations:
Temperature Control: In various production methods, the temperature may be carefully controlled to influence the formation and stability of the nanoemulsion or nanoplatforms. Higher temperatures can reduce the viscosity of the mixture, facilitating the emulsification process, but may also lead to degradation of sensitive components. The method can include precise temperature regulation to optimize the balance between emulsification efficiency and stability.
Pressure Variations: For high-pressure techniques, such as homogenization, the pressure applied during the process can be varied to control the size and distribution of nanoparticles. Higher pressures may lead to smaller particle sizes, but may also increase energy costs and the risk of degrading sensitive compounds. The number of pressure cycles may also be varied to ensure the formation of uniform nanoparticles.
Mixing Speeds and Techniques: In one embodiment, the production method includes mixing the reactants under high shear or vortex conditions to ensure even distribution of particles and prevent agglomeration. The mixing speed and duration can be optimized to achieve the desired particle size and homogeneity. Different types of mixers, such as rotor-stator mixers, may be employed based on the formulation needs.

### Production Process

The method of production may involve multiple stages, with each stage designed to refine the properties of the nanoplatforms or nanoemulsions:
Preparation of the Oil Phase: In this first stage, the oil phase is prepared in a suitable container. This phase includes one or more oils (such as medium or long-chain triglycerides, essential oils, or bioactive lipids), along with lipophilic excipients and any liposoluble active ingredients.
Preparation of the Aqueous Phase: Simultaneously, the aqueous phase is prepared in another container. This phase consists of a buffered aqueous solution (such as phosphate-buffered saline or PBS), along with hydrophilic surfactants and co-emulsifying agents.
Formation of the Preliminary Emulsion: Once both phases have reached the appropriate temperature, preliminary emulsification is carried out by combining the oil phase and the aqueous phase.
Formation of the Nanoemulsion: The droplet size of the preliminary emulsion is reduced to a nanometric range through the application of high-energy homogenization techniques. This step is key to obtaining the desired nanoemulsion.
Cooling: After homogenization, the resulting nanoemulsion undergoes a controlled cooling process to ensure its stability.
Optimization of Nanoparticle Properties: The method of production can also include various optimization steps to ensure that the nanoplatforms or nanoemulsions exhibit the desired properties:
Particle Size Control: Throughout the production process, the size of the nanoparticles may be continuously monitored and adjusted. Techniques such as dynamic light scattering (DLS) or electron microscopy may be used to assess the size and uniformity of the nanoparticles. Adjustments to process parameters such as mixing speed, surfactant concentration, or pressure can be made to achieve the desired size distribution.
Zeta Potential Optimization: The method may involve adjusting the surface charge (zeta potential) of the nanoparticles to enhance their stability in suspension. This can be done by modifying the surfactants, adding charge-inducing agents, or altering the pH of the formulation. A higher zeta potential can prevent particle aggregation, ensuring a longer shelf life for the product.

### Methods of Treatment for Breast Cancer

The disclosure, as illustrated and described herein, is not limited to the details shown because various modifications and changes can be made without departing from the scope of the disclosure and the equivalent of the claims. The following description of specific embodiments will help clarify the construction, operation, and additional benefits of the disclosure.

In this embodiment, the pharmaceutical formulation contains a combination of compounds known as THC (tetrahydrocannabinol), CBD (cannabidiol), CBG (cannabigerol), and CBC (cannabichromene), which are selected for their potential therapeutic benefits in addressing breast cancer. In this embodiment, the method involves administering a single capsule containing 100 mg of the active ingredient daily for a duration of 12 weeks. In this embodiment, the method involves administering an effective concentration of the active principle within the range of 90 mg to 110 mg per day. In this embodiment, the composition comprising nanoplatforms carrying phytocannabinoids is presented in a capsule and administered orally, with a treatment duration of three to six months based on clinical development and laboratory testing.

In this embodiment, the method inhibits the growth of tumor-initiating cells or reduces the likelihood of breast cancer recurrence in a subject undergoing anti-cancer therapy. The method involves assessing the subject for stage I or II breast cancer and, if confirmed, administering an effective amount of this cannabinoid pharmaceutical formulation. In this embodiment, the formulation is designed as a primary treatment option for stage I and II breast cancer. The treatment objectives include reducing tumor size, preventing metastatic spread, inducing apoptosis in cancer cells, and inhibiting tumor angiogenesis.

In this embodiment, the pharmaceutical formulation aims to achieve complete disease regression in breast cancer, targeting underlying molecular pathways involved in tumor progression. In this embodiment, the treatment acts as a Vascular Endothelial Growth Factor (VEGF) inhibitor and an antagonist of tumor angiogenesis, promoting apoptosis in breast cancer cells. In this embodiment, the patient may be female or male, and the age range for the treatment is broad, including patients both under 65 and over 65 years old.

In this embodiment, the treatment targets locally advanced breast cancer that has not spread outside the breast and local lymph nodes. At the time of diagnosis, the cancer may be at stage I or II according to the American Joint Committee on Cancer (AJCC) Cancer Staging Manual (2017). In this embodiment, the breast cancer may include HER2+, ER+, PR+, Triple Negative Breast Cancer (TNBC), ductal carcinoma in situ, invasive ductal carcinoma, lobular carcinoma in situ, or invasive lobular carcinoma. In this embodiment, the cannabinoid composition is administered daily over three to six months, using nanoplatforms to enhance the bioavailability, safety, and efficacy of the compounds.

In this embodiment, the breast cancer being treated could include tumors classified by the TNM system, such as T1, T2, N0, N1, N1mi, and M0. In this embodiment, the patient's breast tumor may range from ≤20 mm to >20 mm but ≤50 mm in greatest dimension, with regional lymph node micrometastasis or metastasis. The treatment is effective in cases where no distant metastases are present.

In this embodiment, the method for treating breast cancer further includes the step of identifying individuals using imaging studies, such as mammography, ultrasound, or MRI. The findings from these imaging studies are then reported as consistent with BI-RADS categories 4, 5, or 6, indicating a higher risk of malignancy. In this embodiment, the method acts as a VEGF antagonist and apoptosis promoter. The dosing regimen consists of administering one capsule per day for six months, with administration exclusively during nighttime hours or 60 minutes prior to meals during daytime hours, depending on the treatment phase.

In this embodiment, the treatment is designed as a monotherapy, neoadjuvant therapy, or adjuvant therapy, depending on the patient's condition. In this embodiment, the treatment method may involve the use of various therapeutic agents, either alone or in combination, to target cancer cells. Single-agent therapies may be employed, using a therapeutic agent such as a chemotherapeutic drug, monoclonal antibody, or a novel compound like cannabinoids.

In one embodiment, combination therapies may be utilized, incorporating chemotherapy, targeted therapies, immunotherapies, or hormone therapies in combination with the cannabinoids. In one embodiment, adjunctive therapies including cannabinoids may also be included to manage side effects and improve patient outcomes. In this embodiment, desired clinical outcomes include alleviating symptoms, reducing tumor size, stabilizing or slowing disease progression, preventing metastasis or recurrence, achieving remission, reducing medication dosage, improving quality of life, and/or prolonging survival.

In various embodiments, methods of treating breast cancer can involve administering pharmaceutical compositions containing active therapeutic agents, such as chemotherapeutic agents, targeted therapies, or novel compounds like cannabinoids. These methods may vary depending on factors such as the stage of the cancer, patient-specific characteristics, and the nature of the composition being administered.

In one embodiment, the treatment method may also vary based on the dosing regimen and schedule, which are critical to balancing efficacy with tolerability. In one embodiment, a single-dose treatment method is used with a high concentration of the therapeutic agent being effective and requiring rapid reduction of tumor burden. This approach may be followed by a maintenance phase with lower doses or alternative therapies.

In other embodiments, the treatment method may involve cyclical dosing, where therapeutic agents are administered in cycles with defined periods of treatment followed by rest periods. This method allows healthy cells to recover between cycles and may reduce cumulative toxicity, making it a common approach in chemotherapeutic regimens.

In one embodiment, continuous or prolonged dosing for cases where sustained suppression of tumor growth is desired may involve continuous or prolonged dosing of the therapeutic agents via slow-release formulations, such as transdermal patches, or through the use of oral formulations taken daily for extended periods.

In one embodiment, the treatment method can be tailored based on the molecular characteristics of breast cancer and patient-specific factors. In one embodiment, targeted therapy based on genetic profile involves the administration of targeted therapies selected based on genetic mutations or molecular markers present in the patient's tumor. This approach may involve the use of drugs that specifically inhibit pathways such as HER2, PI3K, or BRCA.

In one embodiment, therapies targeting hormone receptor-positive breast cancer involve the administration of hormone-blocking agents, selective estrogen receptor modulators (SERMs) or aromatase inhibitors. In one embodiment, therapies targeting immune modulation incorporate immunotherapies, such as immune checkpoint inhibitors, that enhance the ability of the patient's immune system to recognize and destroy cancer cells.

In one embodiment, therapies targeting immune modulation may be used alone or in combination with other therapies, depending on the immunogenicity of the tumor. In one embodiment, methods for monitoring and adjusting the treatment during its administration may include continuous monitoring and dosage adjustments to achieve therapeutic results.

In one embodiment, monitoring of tumor DNA biomarkers or protein markers may involve regular assessment of the values, to assess treatment effectiveness and detect early signs of resistance or recurrence, with real-time adjustments to the treatment regimen, such as switching to alternative therapies if resistance is detected.

In one embodiment, image-based monitoring may include periodic imaging studies, such as MRI, CT, or PET scans, to monitor tumor response to therapy, which may guide continuation, intensification, or modification of the treatment regimen based on changes in tumor size or spread.

In one embodiment, adaptive treatment plans may be designed to be adaptive, with the ability to modify the dose, route of administration, or combination of therapies in response to changes in the patient's condition or tumor behavior to ensure the most effective and personalized care throughout their treatment.

### EXAMPLES

### Examples of Pharmaceutical Compositions

The current example discloses a therapeutic agent aimed at treating breast cancer. This agent comprises a unique composition that utilizes nanoparticles containing cannabinoids, such as THC, CBD, CBG. The formulation is defined by designated percentage ranges of each of these compounds to ensure effective administration.

The example describes a substance designed to treat breast cancer in patients. This substance comprises a formulation that utilizes nanoparticles containing cannabinoids, such as THC, CBD, CBG. Each compound's percentage range for administration is outlined to optimize therapeutic effects. Particular amounts of cannabinoids: 5 mg THC, 35 mg of CBD, 30 mg of CBG, and 30 mg of CBC=100 mg actives per capsule.

### Formulation 1:500 mg Per Capsule

| Component | Quantity (mg) | Percentage (%) |
|---|---|---|
| THC | 5 | 1 |
| CBD | 35 | 7 |
| CBG | 30 | 6 |
| CBC | 30 | 6 |
| Medium chain triglycerides | 179 | 35.80 |
| Sorbitan 80 (Span 80) | 7 | 1.4 |
| Vitamin E | 0.333 | 0.07 |
| Polysorbate 80 (Tween 80) | 7 | 1.4 |
| Polyethylene glycol 400 | 6.66 | 1.33 |
| Phosphate-buffered saline (PBS) | 200.007 | 40 |

### Formulation 2:250 mg Per Capsule

| Component | Quantity (mg) | Percentage (%) |
|---|---|---|
| THC | 5 | 2 |
| CBD | 35 | 14 |
| CBG | 30 | 12 |
| CBC | 30 | 12 |
| Medium chain triglycerides | 60 | 24 |
| Sorbitan 80 (Span 80) | 7 | 2.80 |
| Vitamin E | 0.17 | 0.07 |
| Polysorbate 80 (Tween 80) | 7 | 2.80 |
| Polyethylene glycol 400 | 1.67 | 0.67 |
| Phosphate-buffered saline (PBS) | 74.16 | 29.67 |

### Formulation 3:500 mg Per Capsule

| Component | Quantity (mg) | Percentage (%) |
|---|---|---|
| THC | 5 | 1 |
| CBD | 35 | 7 |
| CBG | 30 | 6 |
| CBC | 30 | 6 |
| Medium chain triglycerides | 179 | 35.80 |
| Sorbitan 80 (Span 80) | 7 | 1.4 |
| Vitamin C | 0.5 | 0.1 |
| Polysorbate 80 (Tween 80) | 7 | 1.4 |
| Polyethylene glycol 400 | 6.66 | 1.33 |
| Purified water | 199.84 | 39.97 |

### Formulation 4:250 mg Per Capsule

| Component | Quantity (mg) | Percentage (%) |
|---|---|---|
| THC | 5 | 2 |
| CBD | 35 | 14 |
| CBG | 30 | 12 |
| CBC | 30 | 12 |
| Medium chain triglycerides | 60 | 24 |
| Sorbitan 80 (Span 80) | 7 | 2.80 |
| Vitamin C | 0.25 | 0.1 |
| Polysorbate 80 (Tween 80) | 7 | 2.80 |
| Polyethylene glycol 400 | 1.67 | 0.67 |
| Purified water | 74.08 | 29.63 |

### Nanoemulsion Process

### Example 1

Oil phase: A quantity of 35.80 grams of medium-chain triglycerides (MCT) is added to a suitable container. Subsequently, 1.40 grams of Sorbitan 80 (Span 80) are incorporated. Immediately, 1 gram of THC, 7 grams of CBD, 6 grams of CBG and 6 grams of CBC is added. The mixture is stirred at a speed of 500 revolutions per minute (rpm) until all components are fully integrated. The oil phase is then heated to a temperature between 40° C. and 45° C.

Aqueous phase: In 40.00 grams of phosphate-buffered saline (PBS), 1.33 grams of polyethylene glycol 400 (PEG 400) and 1.40 grams of polysorbate 80 (Tween 80) are added. The resulting mixture is stirred at a speed of 500 rpm until all ingredients are fully incorporated. The aqueous phase is then heated to a temperature between 40° C. and 45° C.

Formation of the preliminary emulsion: Once both the oil and aqueous phases have reached the established temperature, the oil phase is slowly and gradually added to the aqueous phase, under constant stirring. The mixture is stirred for a period of 30 minutes to ensure the formation of a homogeneous preliminary emulsion.

Formation of the nanoemulsion: To reduce the droplet size in the preliminary emulsion to a range of 50 to 200 nanometers (nm), a high-energy ultrasonic homogenizer is used. The sonication process is carried out in 10-second sonication cycles followed by 10 seconds of rest, for a total period of 15 to 20 minutes. During the entire sonication process, the temperature of the emulsion is maintained at 50° C.

Cooling of the nanoemulsion: After the sonication process is completed, the resulting nanoemulsion is cooled until it reaches a temperature below 30° C., while stirring at a low speed between 30 and 50 rpm, to ensure uniform droplet distribution during cooling.

Capsule filling: Once the nanoemulsion has reached the appropriate temperature, the filling of the capsules is carried out. The nanoemulsion is introduced into soft gelatin capsules.

### Example 2

Oil phase: A quantity of 24.00 grams of medium-chain triglycerides (MCT) is added to a suitable container. Subsequently, 2.80 grams of Sorbitan 80 (Span 80) and 0.07 grams of Vitamin E are incorporated. Immediately, 2 grams of THC, 14 grams of CBD, 12 grams of CBG and 12 grams of CBC is added. The mixture is stirred at a speed of 500 revolutions per minute (rpm) until all components are fully integrated. The oil phase is then heated to a temperature between 60° C. and 70° C.

Aqueous phase: In 29.67 grams of phosphate-buffered saline (PBS), 0.10 grams of vitamin C, 0.67 grams of polyethylene glycol 400 (PEG 400) and 2.80 grams of polysorbate 80 (Tween 80) are added. The resulting mixture is stirred at a speed of 500 rpm until all ingredients are fully incorporated. The aqueous phase is then heated to a temperature between 60° C. and 70° C.

Formation of the preliminary emulsion: Once both the oil and aqueous phases have reached the established temperature, the oil phase is slowly and gradually added to the aqueous phase, under constant stirring. The mixture is stirred for a period of 30 minutes to ensure the formation of a homogeneous preliminary emulsion.

Formation of the nanoemulsion: To reduce the droplet size in the preliminary emulsion to a range of 50 to 200 nanometers (nm), a high-pressure homogenizer is used. The homogenization process is carried out by applying pressures between 1000 and 1500 bar, over 3 to 5 cycles of homogenization. During each cycle, the emulsion is forced through a narrow valve at high speed, generating shear forces that reduce the droplet size. It is crucial to monitor the temperature of the emulsion throughout the process, keeping it between 60° C. and 70° C., using a cooling system if necessary, to preserve the stability of the active ingredients

Cooling of the nanoemulsion: After the sonication process is completed, the resulting nanoemulsion is cooled until it reaches a temperature below 30° C., while stirring at a low speed between 30 and 50 rpm, to ensure uniform droplet distribution during cooling.

Capsule filling: Once the nanoemulsion has reached the appropriate temperature, the filling of the capsules is carried out. The nanoemulsion is introduced into vegetarian capsules.

### Example 3

Oil phase: A quantity of 35.80 grams of medium-chain triglycerides (MCT) is added to a suitable container. Subsequently, 1.40 grams of Sorbitan 80 (Span 80) are incorporated. Immediately, 1 gram of THC, 7 grams of CBD, 6 grams of CBG and 6 grams of CBC is added. The mixture is stirred at a speed of 500 revolutions per minute (rpm) until all components are fully integrated. The oil phase is then heated to a temperature between 40° C. and 45° C.

Aqueous phase: In 39.97 grams of purified water, 0.10 grams of vitamin C, 1.33 grams of polyethylene glycol 400 (PEG 400) and 1.40 grams of polysorbate 80 (Tween 80) are added. The resulting mixture is stirred at a speed of 500 rpm until all ingredients are fully incorporated. The aqueous phase is then heated to a temperature between 40° C. and 45° C.

Formation of the preliminary emulsion: Once both the oil and aqueous phases have reached the established temperature, the oil phase is slowly and gradually added to the aqueous phase, under constant stirring. The mixture is stirred for a period of 30 minutes to ensure the formation of a homogeneous preliminary emulsion.

Formation of the nanoemulsion: To reduce the droplet size in the preliminary emulsion to a range of 50 to 200 nanometers (nm), a high-energy ultrasonic homogenizer is used. The sonication process is carried out in 10-second sonication cycles followed by 10 seconds of rest, for a total period of 15 to 20 minutes. During the entire sonication process, the temperature of the emulsion is maintained at 50° C.

Cooling of the nanoemulsion: After the sonication process is completed, the resulting nanoemulsion is cooled until it reaches a temperature below 30° C., while stirring at a low speed between 30 and 50 rpm, to ensure uniform droplet distribution during cooling.

Capsule filling: Once the nanoemulsion has reached the appropriate temperature, the filling of the capsules is carried out. The nanoemulsion is introduced into soft gelatin capsules.

### Example 4

Oil phase: A quantity of 24.00 grams of medium-chain triglycerides (MCT) is added to a suitable container. Subsequently, 2.80 grams of Sorbitan 80 (Span 80) incorporated. Immediately, 2 grams of THC, 14 grams of CBD, 12 grams of CBG and 12 grams of CBC is added. The mixture is stirred at a speed of 500 revolutions per minute (rpm) until all components are fully integrated. The oil phase is then heated to a temperature between 60° C. and 70° C.

Aqueous phase: In 29.63 grams of purified water, 0.10 grams of vitamin C, 0.67 grams of polyethylene glycol 400 (PEG 400) and 2.80 grams of polysorbate 80 (Tween 80) are added. The resulting mixture is stirred at a speed of 500 rpm until all ingredients are fully incorporated. The aqueous phase is then heated to a temperature between 60° C. and 70° C.

Formation of the preliminary emulsion: Once both the oil and aqueous phases have reached the established temperature, the oil phase is slowly and gradually added to the aqueous phase, under constant stirring. The mixture is stirred for a period of 30 minutes to ensure the formation of a homogeneous preliminary emulsion.

Formation of the nanoemulsion: To reduce the droplet size in the preliminary emulsion to a range of 50 to 200 nanometers (nm), a high-pressure homogenizer is used. The homogenization process is carried out by applying pressures between 1000 and 1500 bar, over 3 to 5 cycles of homogenization. During each cycle, the emulsion is forced through a narrow valve at high speed, generating shear forces that reduce the droplet size. It is crucial to monitor the temperature of the emulsion throughout the process, keeping it between 60° C. and 70° C., using a cooling system if necessary, to preserve the stability of the active ingredients.

Cooling of the nanoemulsion: After the sonication process is completed, the resulting nanoemulsion is cooled until it reaches a temperature below 30° C., while stirring at a low speed between 30 and 50 rpm, to ensure uniform droplet distribution during cooling.

Capsule filling: Once the nanoemulsion has reached the appropriate temperature, the filling of the capsules is carried out. The nanoemulsion is introduced into vegetarian capsules.

1. A composition for treatment of breast cancer, comprising:
a) an active principle; and
b) a stabilizing solubilizing agent;
wherein the active principle comprises a cannabinoid.

2. The composition according to claim 1, wherein the cannabinoid is present in a concentration in the range of 20% to 40%, and the stabilizing solubilizing agent is present in a concentration in the range of 0.1% to 5%.

3. The composition according to claim 2, wherein the cannabinoid comprises one or more of the following: tetrahydrocannabinol (THC), cannabidiol (CBD), cannabigerol (CBG), or cannabichromene (CBC).

4. The composition according to claim 3, wherein THC is present in a concentration in the range of 1.00-2.00%, CBD in the range of 7.00-14.00%, CBG in the range of 6.00-12.00%, and CBC in the range of 6.00-12.00%.

5. The composition according to claim 1, wherein the active principle is incorporated into a nanoplatform formulation comprising nanoparticles.

6. The composition according to claim 1, wherein the stabilizing solubilizing agent is polyethylene glycol PEG-400.

7. The composition according to claim 1, wherein the nanoparticles have an average size of less than 200 nm.

8. The composition according to claim 1, wherein the nanoplatform formulation is designed to provide a controlled release of cannabinoids over time.

9. The composition according to claim 1, wherein the formulation is suitable for oral administration and is provided in capsule form.

10. A method for treating breast cancer in a human individual, comprising administering an effective concentration of the composition of claim 1, wherein the active principle comprises a cannabinoid and the stabilizing solubilizing agent.

11. The method according to claim 10, wherein the human individual is selected for treatment based on being diagnosed with stage I or stage II breast cancer.

12. The method according to claim 10, wherein the individual is selected for treatment based on immunohistochemistry results for at least one of the following: estrogen receptor-positive (ER+), progesterone receptor-positive (PR+), HER2-positive breast cancer or TNBC.

13. The method of claim 11, further comprising identifying individuals using imaging studies such as mammography, ultrasound, or MRI, and reporting findings consistent with BI-RADS categories 4, 5, or 6.

14. The method of claim 11, wherein the individual is identified by having elevated serum biomarkers, including CA153 levels above 30 U/mL and CA2729 levels above 38 U/mL.

15. The method according to claim 10, wherein the effective concentration is administered as a single capsule containing 100 mg of active principle daily for a period of 12 weeks.

16. The method of claim 15, wherein the effective concentration of the active principle is in the range of 90 mg to 110 mg per day.

17. The method according to claim 10, wherein the administering of the effective concentration of the composition acts as a monotherapy for treating breast cancer stages I and II.

18. The method of claim 10, wherein the treatment composition inhibits tumor angiogenesis by targeting VEGF pathways and VEGF receptors overexpressed in breast cancer cells.

19. A method for producing a pharmaceutical composition for treating breast cancer, comprising: incorporating a cannabinoid into a nanoplatform formulation comprising a stabilizing solubilizing agent, wherein the formulation is suitable for oral administration, and wherein further comprising preparing the cannabinoids using nano emulsification techniques to ensure the formation of nanoparticles with a size less than 200 nm.

20. A cannabinoid-based pharmaceutical composition comprising:
Tetrahydrocannabinol (THC) in a concentration ranging from 1% to 2%;
Cannabidiol (CBD) in a concentration ranging from 7% to 14%;
Cannabigerol (CBG) in a concentration ranging from 6% to 12%;
Cannabichromene (CBC) in a concentration ranging from 6% to 12%; and
Polyethylene glycol 400 (PEG 400) in a concentration ranging from 0.5% to 1.5%;
wherein the composition is formulated as a nanoplatform to enhance the bioavailability and therapeutic efficacy of the cannabinoids for treating cancer.
